(19) United States
(12) Patent Application Publication
(10) Pub. No.: US 2013/0012956 A1
(43) Pub. Date: Jan. 10, 2013

(54) INTRAOCULAR LENS INJECTING DEVICE

(76) Inventor: Ram Srikanth Mirlay, Karnataka (IN)

(21) Appl. No.: 13/635,269

(22) PCT Filed: Mar. 7, 2011
(86) PCT No.: PCT/IN2011/000146

§ 371 Date: Sep. 14, 2012

(30) Foreign Application Priority Data
Apr. 22, 2010 (IN) 1135/CHE/2010

(51) Int. Cl. A61F 9/013; A61F 2/16

(52) U.S. Cl. 606/107

(57) ABSTRACT

The embodiments herein provide an intra-ocular lens injecting device for implanting the same through an incision into an eye of a subject. The device comprises a cartridge for loading at least one intra ocular lens and a delivery tube operably coupled to the cartridge for delivery of the intra ocular lens through the incision. The delivery tube has a rigid first section and a flexible second section. The rigid first section and the flexible second section are configured to have a uniform and a substantially oval cross-sectional area. The configuration of the delivery tube reduces trauma and the incision size, to the subject during implantation. The device also includes a plurality of projections provided on the flexible second section of the delivery tube.

### BACKGROUND

1. Technical field

The embodiments herein generally relate to an intraocular lens injecting device and more particularly to a delivery tube of the intraocular lens injecting device.

2. Description of the Related Art

An intraocular lens, referred to hereinafter as IOL, is a Tens implanted in the eyes of any mammal, the behavior of which is predominantly vision based, including humans. The implantation of the IOL is a replacement for the defective crystalline eyes preexisting in the animal/human. The defect can be formation of a cataract or increase/decrease/imperfection/incorrectness in the eye's optical power. The IOL is a small plastic lens with plastic side struts/supports, called haptics, to hold the lens in place within a capsular bag inside the eye. The IOLs are foldable and are implanted into the eyes through an incision in the eye. The length of the incision made is usually smaller than the diameter of the IOL to be implanted. Normally, in order to implant an IOL through an incision, an IOL injecting apparatus is employed.

Conventional IOL injecting apparatus include a hollow insertion tube having a diameter that allows free passage of the foldable IOL without undergoing any permanent deformation. An IOL loading and retaining mechanism, referred to commonly as cartridge is also provided in certain injecting apparatus to hold, fold and inject into the eye, a plurality of IOLs. Further, the IOLs injected can include IOLs of various sizes, designs, powers, materials and color. A plunger is provided to push the IOL that is transferred from the cartridge into a delivery channel, also referred to as tube, or the insertion tube. FIG. 1 shows the cross-sectional profiles of a delivery tube 107 according to a known IOL injector apparatus in an incision 116 cut into an eye 114. Normally, the insertion tubes have a predominantly circular cross-sectional area 115 at perpendicular to long axis of the IOL injector apparatus. However, the incision 116 made into the eye has a non-circular cross sectional area profile 115 during instrumentation and surgical manipulations. The tip of the delivery tube 107 has to penetrate the incision for proper delivery of the IOL.

Introduction of the insertion tube through the incision of the eye 114 to implant the IOL causes undue excessive expansion of the incision due to a mismatch in the corresponding cross sectional areas and shapes resulting in increased stretch trauma to the eye. This forces a surgeon to increase the incision size 117 and 118, to reduce the incision stretch pressure. Modern cataract surgery is performed on the non-immobilized eye and there is a risk of inadvertently excessive surgical entry and eye damage with the known injector apparatus designs. Hence, there is a need for an IOL injecting apparatus that substantially reduces the stretch-trauma caused during the insertion of the IOL and the incision size made during corrective surgical procedures.

Therefore, there is a need for an IOL injecting device, which can reduce the trauma and the incision size, while injecting the IOL into an eye of a subject.

### SUMMARY

The embodiments herein provide an intraocular lens injecting device for implanting an IOL in a subject. The device comprises a cartridge for loading at least an intra ocular lens and a delivery tube operably coupled to a cartridge for delivery of the IOL. The delivery tube has a first section configured to form a rigid structure and a second section configured to form a flexible structure. The rigid structure and the flexible construction are configured to have an identical oval cross-sectional area, at perpendicular to long axis of the delivery tube. The oval cross-sectional configuration of the delivery tube significantly reduces trauma, and the incision size, to the subject during implantation of the IOL. The delivery tube also includes a plurality of safety-projections provided on the flexible structure of the delivery tube.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

FIG. 1 shows cross-sectional profiles of a delivery tube of a known IOL injector device injected through an incision of an eye of a subject.

FIG. 2 is a perspective view of an IOL injection device with a delivery tube of the present invention.

FIG. 3 shows first and second sections of the delivery tube having ribs and webs according to one embodiment herein.

FIG. 4 is a perspective view of the delivery tube of the present invention in a normal and empty state.

FIG. 5 shows the delivery tube in an open state, with a folded IOL disposed in the delivery tube during the course of passage of the IOL through the delivery tube.

FIG. 6 shows the cross sectional view of the delivery tube, according to one embodiment herein, with uniform cross section across the length of the delivery tube.

FIG. 7 shows cross-sectional profiles of the delivery tube of the IOL injector device of the present invention along with an incision cut into an eye of a subject.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

Various embodiments herein provide an IOL injecting device. More particularly, specific embodiments herein provide an IOL injecting device with an improved construction having enhanced safety features.

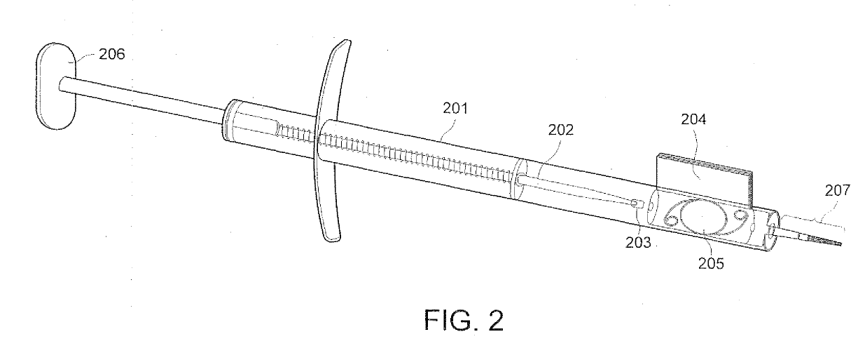

The IOL injecting device with a delivery tube 207 of the present invention as shown in FIG. 2 includes a cylindrical-shaped injector body 201 with a longitudinal inner space. A movable plunger 202 with a plunger tip 203, operable by a surgeon, with a thumb press 206, is disposed inside the space of the cylindrical body 201 for engaging, advancing and delivering an intraocular lens 205, into an eye of a subject, at the time of an ophthalmic surgical procedure. The delivery tube 207 with a rigid first section and a flexible second section, the constructional features of which are more fully described hereinafter, is connected to the injector body 201. A cartridge 204 with a lens holding gutter is connected to the delivery tube 207, as shown in FIG. 2, for holding, folding and releasing an IOL 4, into the delivery tube 207 at the time of surgical operation. The IOL injecting device as shown in FIG. 2 is exemplary in nature to depict the connectivity of the delivery tube 207 of the present invention to the injector body 201. It is to be understood that the delivery tube 207 of the present invention can be adapted to any other suitable IOL injecting devices that are used in ophthalmic surgical procedures.

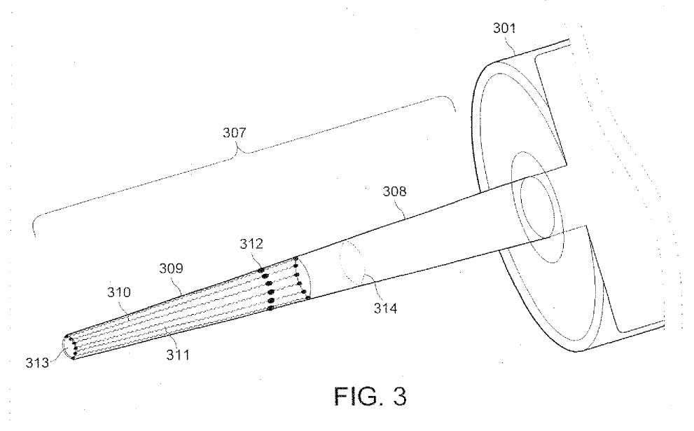

FIG. 3 shows the delivery tube according to an embodiment herein. The delivery tube 307, which is connected to the injector body 301 includes a rigid first section 308, which is cylindrical and configured to have a substantially oval cross-sectional profile 314, at perpendicular to the long axis of the delivery tube 307. The rigid first section 308 is connected to the injector body 301 and the cartridge 203 to hold and receive the IOL 205 (as shown in FIG. 2), for a subsequent advancement and implantation into the eye of the selected subject. The IOL 205 as received by the rigid first section 308, from the cartridge 204, undergoes a temporary deformation into a folded and compressed configuration, while being advanced through the delivery tube 307, with the actuation of the movable plunger 202, which engages and advances the IOL 205.

A second section 309 is connected to the rigid first section 308 of the delivery tube 307. The second section 309 is provided with a flexible construction, as hereinafter described.

The flexible second section 309 is provided with a plurality of rigid ribs 310, extending longitudinally along the long axis of the delivery tube 307 as shown in FIG. 3. The rigid ribs 310 are disposed to expand or stretch upon application of pressure by the folded IOL 205, while the IOL 205 advances through the flexible second section 309, when the IOL 205 is engaged by the movable plunger or the piston 202. As an exemplary embodiment, about 4-10 ribs 310 are shown anchored to the rigid first section 308. The junction points of the ribs 310 form an oval shape and terminate as a self-adjustable tip 313, which is elastic in nature. The self-adjustable tip 313 is arranged to expand during the course of delivery of the folded IOL 205 through the self-adjustable tip 313. The aforementioned anchoring of the ribs 310 is achieved by any known method of anchoring, which includes presently used methods, but is not limited to riveting, molding, screwing, adhesion etc. A sheet of foldable, non-elastic plastic of surgical grade, sterilizable material connects each of the rigid ribs 310, to form webs 311, between the rigid ribs 310. In an example of the embodiments herein, the rigid first section 308 and the flexible second section 309 of the delivery tube 307 is formed in a single process to possess a corresponding rigid and flexible construction. The web of plastic 311 starts in continuation of lumen of the loading and folding bay's lens-holding gutter connected to the cartridge 204 (as shown in FIG. 2). The outer dimensions of the delivery tube 307 are in the range of 3 to 9 mm in the horizontal plane and in the range of 2 to 5 mm in the vertical plane, for human eye lenses. These dimensions vary for the veterinary use, depending on the size of the desired mammalian eye. The flexible second section 309 is also provided with knob-like projections 312 as more fully described hereinafter.

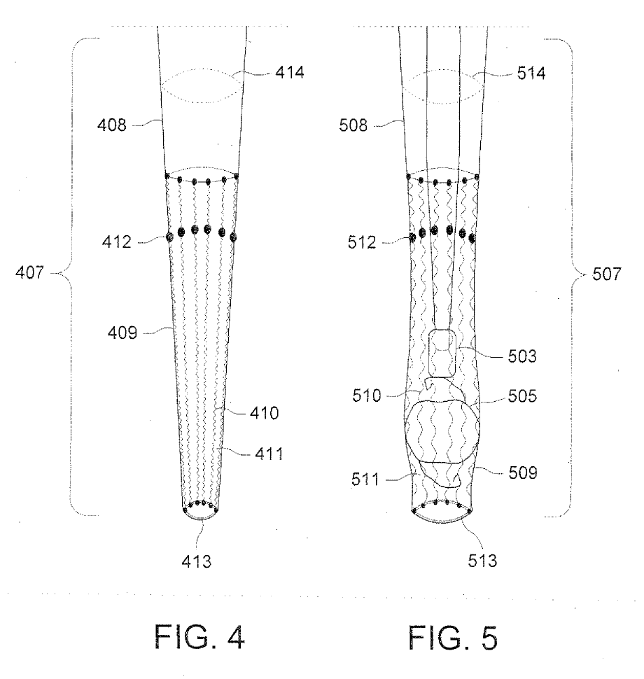

FIG. 4 shows the delivery tube 407 with rigid first section 408 and second flexible section 409, in a closed or normal state, according to another embodiment herein. The delivery tube 407 is provided with a substantially oval cross section 414. In the closed state, the rigid ribs 410 of the flexible second section 409 with the knob-like projections 412 converge to a total vertical and horizontal size of about 0.5 mm. The rigid ribs 410 are joined together by thin, foldable, inelastic web of plastic 411. The size of each of the segment of web 411 is decided by the manufacturer and based on the compressible size of its IOL. This size determines the maximum expandable size of the IOL. Further, the outer surfaces of the rigid ribs 410 are provided with plurality of knob-like safety projections 412, at a pre-determined distance from the self-adjustable tip 413 of the delivery tube 407. The self-adjusting tip 413 is elastic, expandable and has an oval-shaped cross section taken at perpendicular direction to the long axis of tube 407. The self-adjusting tip 413 is joined with the rigid ribs 410.

FIG. 5 shows the delivery tube 507, having substantially oval cross-section 514 with rigid first section 508 and flexible second section 509, along with the knob-like projections 512, in an expanded or open configuration according to an embodiment herein. In an open configuration, the ribs 510 are in an extended state. The extended state is also referred to as pushed apart state or dilated delivery tube state, all of which refers to the same embodiment referred to and described herein. The pressure created by the moving tip 503 of plunger, pushes the loading and folded IOL 505 through the rigid first section 205 (FIG. 2) of the delivery tube 507 from the loading and folding bay of the cartridge 204 (FIG. 2) towards into the flexible second section 509. The abutment of the moving IOL 505 with the inner surface or walls of the flexible second section 509 facilitates the extension of the rigid ribs 510 resulting in the unfolding or expansion of the web 511. The ribs 510 are configured to expand to the dimensions of the rigid first section 508 of the delivery tube 507. FIG. 5 also depicts the stretching of the rigid ribs 510 and the web 511 in open position of the delivery tube 507, and the expanded self-adjusting tip 513, to facilitate an easy delivery of the IOL 505.

In one embodiment herein, while specifically referring to FIG. 4, the knob-like projections 412 are positioned at a distance in the range of 6 to 10 mm behind the self-adjustable 413 tip of the delivery tube 407. The round knob-like projections 412 prevent accidental surgical over-entry into the eye of the subject and the resultant eye injury during surgery, which is more fully described hereinafter. This is needed because most cataract surgeries are done on moving eyes.

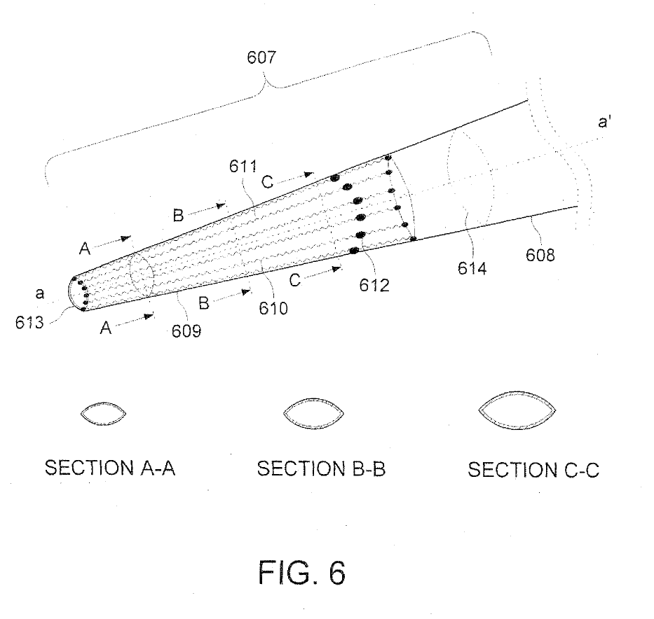

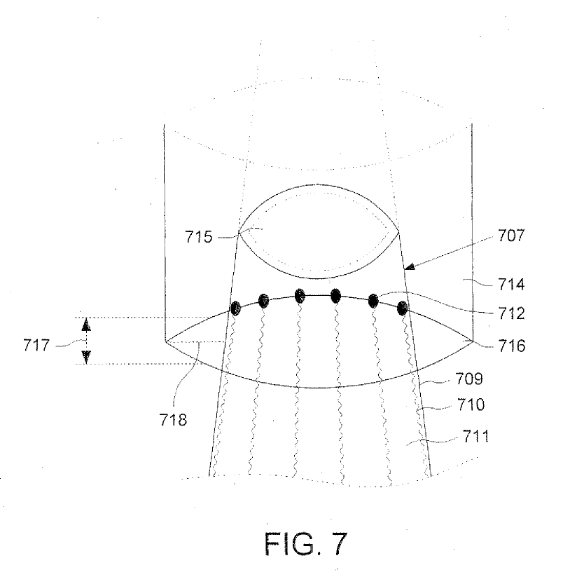

FIG. 6 shows the cross-sectional view of the delivery tube 607, according to an embodiment herein, with uniform cross section across the length of the delivery tube 607. The delivery tube 607 includes a rigid first section 608 and a flexible second section 609. The delivery tube 607 has a substantially oval cross section 614 at perpendicular to the long axis a-a′ of the delivery tube 607. Further, the substantially oval cross section 614 is uniform at various sections A-A, B-B and C-C taken along the length of the delivery tube 607. The uniformly oval cross-section ensures that the folded or temporarily deformed IOL 205 (FIG. 2) retains the same configuration through the journey in the delivery tube and also utilizes the internal space of the incision optimally, as shown in FIG. 7.

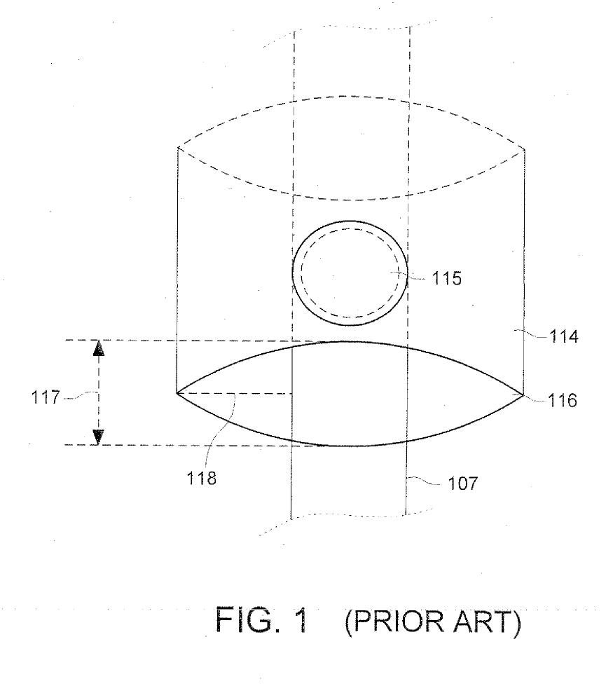

FIG. 7 shows the cross sectional profiles of the delivery tube 707 with second flexible section 709, inserted into an eye 714 of subject through the incision 716 cut into an eye 714. The substantially oval cross section 715 of the delivery tube 707 with rigid ribs 710 and webs 711 almost coincides with the oval incision profile 716 made on the eye of a subject, thereby eliminating the mismatch as shown in FIG. 1 and significantly reducing the trauma to the eye of the subject. It also permits a smaller incision size 718 and 717, for a given IOL size. The configuration of the delivery tube as explained herein before and as illustrated in FIG. 7 eliminates the mismatch in the cross sectional areas and hence greatly reduces the trauma caused to the eye of the subject and optimizes the use of surgical space, and minimizes the incision size 717 and 718. It enables a smaller incision for a given size of the lens when compared to the known injector tubes. The round knob-like projections 712 as shown in FIG. 7 prevent accidental surgical over-entry into the eye of the subject and the resultant eye injury during surgery.

The IOLs may be delivered by the manufacturer, separate from the IOL injecting device, or may be pre-loaded in the IOL, injecting device. The delivery tube of the IOL injection device of the present invention is applicable to separately loaded and pre-loaded IOL injecting devices. In one embodiment herein, the IOL is removed from its sterile packaging, in the operation room, during surgery. It is transferred to the opened cartridge, placed in the loading-bay trench or gutter and then the cartridge is closed. The closing action folds the IOL, compresses it and the cartridge is loaded into the IOL injecting device. The plunger of the IOL injecting device is advance& till it reaches the IOL. The flexible second section of the delivery tube is then introduced into the eye through the incision made. The plunger is advanced to push the folded IOL into the delivery tube. The folded compressed IOL move through the delivery tube pressing outwards and tends to expand the flexible second section of the delivery tube. The rigid ribs expand outwardly up to their limits, press against the incision wail, and guide the IOL into the eye. The self-adjustable tip of the delivery tube, which has a resting aperture of 0.5 mm, row expands to allow the IOL to pass through. The maximum expansion of the self-adjustable tip's aperture is determined by the size of the surgical incision. This feature therefore, auto-adapts to different sizes of IOLs as, well as different sizes of surgical incision. The flexible opening of the delivery tube significantly reduces the trauma caused to the eye due to elimination of the mismatch between the oval cross-section of the incision made and the delivery tube of the IOL injector device. Further, the knobs/ribs provided at the proximity or the opening of the delivery tube ensure safety by preventing the IOL injecting device from being inserted deeply leading to probable damage of the eye.

The invention, as described herein and as illustrated by the drawings provide an improved intra ocular lens injecting apparatus with an uniform and a substantially oval cross-sectional area which significantly reduces the trauma to the subject by eliminating the mismatch between the cross-section of the incision and the cross-section of the delivery tube.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed, embodiments. It is to be understood that the phraseology or terminology employed herein is for the purpose of description and not of limitation.

Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. An intraocular lens injecting apparatus for implanting the same through an incision made into an eye of a subject, the apparatus comprising:
a cartridge for loading at least one intra ocular lens; and
a tube operably coupled to the cartridge for delivery of the intra ocular lens onto the eye of the subject
wherein the tube has a first section configured to form a rigid structure and a second section configured to form a combination of flexible and rigid structure to provide a self adjustable tip to eliminate trauma to a subject during implantation.

2. The apparatus according to, claim 1, further wherein the tube has a uniform and a substantially oval cross section along the length.

3. The apparatus according to claim 1, wherein the tube is formed as a single structure having a rigid section and a flexible section.

4. The apparatus according to claim 1, wherein the cartridge is mounted on an oval cross sectional groove provided in the first section of the tube.

5. The apparatus according to claim 1, wherein the first section of the tube is provided with a plunger to drive the intra ocular lens through the tube subsequent to the release of the same from the cartridge.

6. The apparatus according to claim 1, wherein the second section is provided with a plurality of collapsible ribs capable of expanding upon application of pressure through the plunger.

7. The apparatus according to claim 1, wherein the ribs are positioned in a spaced out relation on the outer surface of the flexible section of the tube.

8. The apparatus according to claim 1, wherein the outer surface of the ribs are provided with plurality of safety projection knobs at a predetermined distance from the second end of the tube to form an annular ring structure.

9. The apparatus according to claim 8, wherein the predetermined distance is 5 to 8 mm.

10. The apparatus according to claim 1, wherein the annular ring structure formed out of the plurality of projections allows restricted entry of the injector apparatus through the incision made into the eye of the subject.

11. The apparatus according to claim 1, wherein the rigid ribs are sheathed with loose pleated plastics of surgical grade to permit an expansion of a lumen during a passage and an extrusion of an intraocular lens.

12. The apparatus according to claim 1, wherein an elastic ring is provided at one end so that all the rigid ribs are terminated at the elastic ring.
